(19) United States
(12) Patent Application Publication
(10) Pub. No.: US 2022/0040396 A1
(43) Pub. Date: Feb. 10, 2022

(54) DEVICE FOR AN ASPIRATOR, ASPIRATOR AND METHOD

(71) Applicant: EXCITUS AS, Stavanger (NO)

(72) Inventors: Øyvind TJØLSEN, Stavanger (NO); Per Reidar ØRKE, Hafrsfjord (NO)

(73) Assignee: EXCITUS AS, Stavanger (NO)

(21) Appl. No.: 17/299,354

(22) PCT Filed: Dec. 4, 2018
(86) PCT No.: PCT/EP2018/083514

§ 371 Date: Jun. 3, 2021

(51) Int. Cl. A61M 1/00

(52) U.S. Cl. CPC A61M 1/774; A61M 1/74; A61M 2205/7563; A61M 1/88; A61M 1/784

(57) ABSTRACT

A device for an aspirator, the device comprising a suction inlet for suctioned air, liquid and particles; an exhaust outlet for air; a reservoir for collecting liquid and particles separated from the air; and a clearing arrangement fluidly between the suction inlet and the exhaust outlet, the clearing arrangement being configured to provide a path of air, substantially cleared from liquid sucked through the suction inlet, to the exhaust outlet, in any orientation of the device in space. An aspirator comprising a device, and a method of modifying a device for an aspirator, are also provided.

### TECHNICAL FIELD

The present disclosure generally relates to a device for an aspirator. In particular, a device for an aspirator, which device comprises a clearing arrangement, an aspirator comprising a device, and a method of modifying a device for an aspirator, are provided.

### BACKGROUND

A medical aspirator, also referred to as a medical suction unit, is a device for to removing e.g. bodily fluids during medical procedures or emergency situations. For example, a patient or victim may need to be exposed to vacuum suction to remove bodily fluids and secretions from the upper airways, lungs or other locations. A medical aspirator is a part of the standard equipment in most ambulances.

Aspirators are also applicable to non-medical uses, such as to remove liquid and/or debris from a confined space. One example is the use of an aspirator in ventilation systems such as heating and cooling systems.

Many aspirators comprise a canister for collecting the received fluids and secretions that needs to be positioned on a horizontal surface in order to function. The aspirator typically also comprises an exhaust air filter in an upper part of the canister, a vacuum pump and a valve arrangement. The filter may be used to prevent particles and liquid droplets from entering the vacuum pump and the valve arrangement. The filter can thereby improve functionality, and reduce cleaning requirements, of the vacuum pump and the valve arrangement. The filter also prevents contaminated particles and liquid droplets from being discharged to the ambient environment.

When the canister is horizontal, the suctioned liquid is collected in the bottom of the canister and the risk of filter clogging is reduced. However, if the canister is not horizontal, the filter might clog rapidly and the suction operation is consequently deteriorated or stopped.

Aspirators of the above type therefore cannot be used in the way most practical and efficient for the user, such as a paramedic. In case the aspirator comprises a hose between the canister and a suction inlet for use on a patient, operation controls (e.g. buttons, knobs and sliders) on the canister are separated from the suction inlet. In this case, a two-hand operation is required, which is not desired.

### SUMMARY

One object of the present disclosure is to provide a device for an aspirator, which device enables a more practical, more efficient and/or simpler use of the aspirator.

A still further object of the present disclosure is to provide a device for an aspirator, which device enables a more reliable operation of the aspirator.

A still further object of the present disclosure is to provide a device for an aspirator, which device provides a longer lifetime and/or reduced requirements of maintenance of the aspirator.

A still further object of the present disclosure is to provide a device for an aspirator, which device has a simple and/or cheap design.

A still further object of the present disclosure is to provide a device for an aspirator, which devices solves several or all of the foregoing objects in combination.

A still further object of the present disclosure is to provide an aspirator comprising a device, which aspirator solves one, several or all of the foregoing objects.

A still further object of the present disclosure is to provide a method of modifying a device for an aspirator, which method solves one, several or all of the foregoing objects.

According to one aspect, there is provided a device for an aspirator, the device comprising a suction inlet for suctioned air, liquid and particles; an exhaust outlet for air; a reservoir for collecting liquid and particles separated from the air; and a clearing arrangement fluidly between the suction inlet and the exhaust outlet, the clearing arrangement being configured to provide a path of air, substantially cleared, or cleared, from liquid sucked through the suction inlet, to the exhaust outlet, in any orientation, or substantially any orientation, of the device in space.

The clearing arrangement may for example be configured to provide the path of air both in a first orientation of the device, and in a second orientation of the device, wherein the device in the second orientation is tilted from the first orientation at least 30 degrees, such as 30 degrees to 8o degrees, about a horizontal axis. In this case, the first orientation may be a horizontal orientation. The device may be said to be horizontal when a center axis of a discharge opening of the suction inlet, e.g. into the reservoir, is horizontal. The clearing arrangement may be configured to provide the path of air substantially independently of, or independently of, gravity force.

Liquids sucked by the aspirator may for example include various types of bodily fluids, such as blood, secretions, gastric, vomit and/or urine. Further examples of sucked liquids may be alcohols, acids, soaps, poisons etc. Particles sucked by the aspirator may for example include sand, gravel, debris and/or bone fragments.

The clearing arrangement according to the present disclosure is not a filter. According to some variants, the clearing arrangement is configured to reduce the presence of liquid in an already existing path to the exhaust outlet, for example from the suction inlet, through the reservoir, and to the exhaust outlet. According to some variants, the clearing arrangement is configured to change an already existing path to the exhaust outlet.

The device may further comprise a filter fluidly between the suction inlet and the exhaust outlet. In this case, the clearing arrangement may be fluidly between the suction inlet and the filter, and the clearing arrangement may be configured to provide a path of air, substantially cleared, or cleared, from liquid, sucked through the suction inlet, to the filter, in any orientation of the device in space. The filter may be provided upstream of a vacuum pump.

The device may further comprise a filter chamber and the filter may be to housed within the filter chamber. The device may further comprise one or more passages for providing air communication between the reservoir and the filter chamber, for example one or more openings in a wall of the filter chamber. The filter may for example be a HEPA (high efficiency particulate arresting) filter. Alternatively, or in addition, the filter may be hydrophobic. The type of filter used may depend on the specific implementation, e.g. may be selected in dependence of which fluids the filter will be exposed to.

The filter may be removable from the device. In some emergency situations, where either the filter is clogged, or the amount of collected fluids exceeds the collectable volume of the device, the filter may be removed and suction operation can be continued without the filter. In such a situation, fluid and droplets may pass through the pump and the valve arrangement, and finally be evacuated out of the device, the result being an increased risk for functional and performance deterioration, in addition to a risk of spreading contamination. But especially in military applications, it would represent one extra last chance of saving a life.

The device may further comprise a distribution structure configured to distribute suctioned liquid into smaller droplets. The distribution structure may be constituted by a wall, e.g. substantially perpendicular, or perpendicular, to a center axis of a discharge opening of the suction inlet, e.g. into the reservoir. The wall of the distribution structure may be a wall of the filter chamber, or another wall within the device.

The clearing arrangement may be movable and/or configured to change in shape. The clearing arrangement may for example be movable within the device, such as within the reservoir.

The clearing arrangement may be configured to separate liquid from air by attracting liquid. The clearing arrangement may thus be configured to demobilize liquid in order to provide the path for air. Several ways exist for attracting liquid, for example by means of static electricity, absorption and/or adsorption.

The clearing arrangement may be configured to separate liquid from air by means of absorption. To this end, the clearing arrangement may comprise one or more substances configured to bind water and/or other liquids.

The clearing arrangement may comprise an absorbent material. One example of a suitable absorbent material is superabsorbent polymers, e.g. a compound which is comprised in the class of superabsorbent polymers. As used herein, “superabsorbent polymers” is material which in contact with water forms hydrogel, thus absorbing and retaining, at least 200%, such as up to 300%, by weight of water. This class of compounds may substantially comprise polymers able to bond the water molecules by means of hydrogen bond. The superabsorbent polymers, or an alternative absorbent material, may be provided in powder form or as particles. According to one variant, the clearing arrangement comprises superabsorbent polymers configured to absorb and retain 0.1-0.5 l water such as 0.2-0.3 l water, for example in 1-3 s. An alternative example of an absorbent material is silica gel.

The clearing arrangement may comprise at least one carrier containing the absorbent material, such as superabsorbent polymers. According to one example, the one or more carriers are constituted by permeable bags. The at least one carrier may be fixed with respect to the device, e.g. to an interior surface of the reservoir, or be movable with respect to the device, e.g. within the reservoir.

Alternatively, or in addition, the clearing arrangement may comprise a partition wall defining a partition volume, e.g. within the reservoir. In this case, the absorbent material, such as superabsorbent polymers, may be provided within the partition volume. Thereby, when liquid enters the partition volume, the swelling of the absorbent material can be controlled. That is, the swelling of the absorbent material can be limited by the partition volume. The partition wall may be permeable, e.g. of the same type as a to coarse filter as described herein.

Alternatively, or in addition, the absorbent material, such as superabsorbent polymers, may be provided in or on at least one free body according to the present disclosure. In this case, the free body may comprise a permeable wall, e.g. such that liquid can enter the body and come in contact with the absorbent material, and such that larger particles are held away from the absorbent material. The permeable wall may for example be a wall comprising holes, or a body made entirely or partly of a mesh etc.

Alternatively, or in addition, the absorbent material may be held at specific locations, such as at the interior surface of the reservoir, by means of static electricity and/or adhesive.

Alternatively, or in addition, the clearing arrangement may be configured to separate liquid from air by means of capillary absorption. To this end, the clearing arrangement may comprise at least one capillary structure. The capillary structure may for example comprise a plurality of capillaries and/or a plurality of parallel plates with small distances between the plates.

In any case, the capillary structure may be either fixed or movable with respect to the device. According to one variant, the capillary structure is attached to an interior surface of the reservoir. For example, the capillary structure may comprise a plurality of parallel plates that protrude, e.g. radially, from the interior surface.

According to a further variant, the capillary structure is provided in one or more movable bodies, such as blocks, for example movable within the reservoir. Each block may for example comprise a capillary structure in the form of capillaries and/or parallel plates. The one or more blocks may be substantially rigid, or rigid. Alternatively, the one or more blocks comprising a capillary structure may be flexible, e.g. configured swell when absorbing liquid.

According to a further variant, the capillary structure comprises a plurality of freely movable bodies, such as balls. A capillary action will then take place in the interstices between the movable bodies. Such movable bodies may for example be freely movable within the reservoir. Due to the gravity force, the liquid and the movable bodies will be positioned in the same region of the device and the liquid will be absorbed by the capillary action in this region. This also applies when the capillary structure comprises a movable block as mentioned above.

The clearing arrangement may comprise an adhesive. The adhesive may for example be provided as a double-sided tape attached to the interior surface of the reservoir or be sprayed onto the interior surface of the reservoir. The adhesive may also be provided on the one or more free bodies according to the present disclosure. In case the device comprises a coarse filter, adhesive may be provided downstream of the coarse filter. The adhesive may for example be used to attract liquid by providing a sticky surface to which for example wet tissue can adhere. Alternatively, or in addition, the adhesive may be used for adhesion of an absorbent material, e.g. in powder form. Attraction by means of adhesion may constitute one example of adsorption.

The clearing arrangement may comprise a cyclone separator. The cyclone separator may be arranged either within the reservoir or outside the reservoir.

The clearing arrangement may comprise at least one free body, such as a plurality of free bodies. The free bodies may be movable within the device, e.g. within the reservoir. The free bodies may for example comprise or constitute a capillary structure, and/or comprise any combination of adhesives or superabsorbent polymers. The free bodies may be substantially rigid, rigid or flexible.

The clearing arrangement may be positioned within the reservoir. However, the clearing arrangement may alternatively be provided outside the reservoir, e.g. in a chamber upstream or downstream of the reservoir, or parallel with the reservoir.

The clearing arrangement may comprise a floating element configured to to float on collected liquid within the reservoir, and a tube having a tube inlet and a tube outlet, and wherein the tube inlet is connected to the floating element and the tube outlet is arranged downstream along the path. The tube inlet can thereby be held above the surface of the liquid within the reservoir. The tube outlet may for example be connected to an opening in a wall of the filter chamber. In this case, only one opening may be provided in the wall of the filter chamber such that all air has to flow through the tube in order to reach the exhaust outlet. In this variant, the path for air passes through the tube. The tube inlet may be provided with a device for preventing liquids, such as droplets, from entering the tube. Such device may for example be a mechanical shield, a coarse filter, or a hydrophobic filter. This type of clearing arrangement comprising a floating element is configured to change the path or air, e.g. as a liquid surface level moves within the reservoir.

The device may further comprise a secondary clearing arrangement, i.e. in addition to the above described primary clearing arrangement, and a switching mechanism; wherein the switching mechanism is configured to switch the secondary clearing arrangement from an inactive state, in which the secondary clearing arrangement is fluidly disconnected from the suction inlet, to an active state, in which the secondary clearing arrangement is arranged fluidly between the suction inlet and the exhaust outlet, and wherein the secondary clearing arrangement is configured to provide a path of air, substantially cleared, or cleared, from liquid sucked through the suction inlet, to the exhaust outlet, in any orientation of the device in space. The secondary clearing arrangement may for example comprise superabsorbent polymers or an alternative absorbent material.

The switching mechanism may be configured to switch the secondary clearing arrangement from the inactive state to the active state by moving the secondary clearing arrangement from an isolated position to an exposed position. The isolated position may be a position where any fluid from the suction inlet is prevented from reaching the secondary clearing arrangement. The exposed position may be a position where the fluid from the suction inlet is guided to the secondary clearing arrangement. Each of the isolated position and the exposed position may for example be provided within the reservoir.

Alternatively, the switching mechanism may be configured to switch the secondary clearing arrangement from the inactive state to the active state by redirecting a fluid flow from the suction inlet. For example, the primary clearing arrangement may be arranged in a primary reservoir and the secondary clearing arrangement may be arranged in a secondary reservoir. The switching mechanism may then be configured to redirect a fluid flow from the suction inlet into the primary reservoir, to a fluid flow from the suction inlet into the secondary reservoir. Thereby, also the secondary clearing arrangement is configured to provide the path of air, substantially cleared from liquid sucked through the suction inlet, to the exhaust outlet, in any orientation of the device in space.

The device according to the present disclosure may comprise a coarse filter in addition to the clearing arrangement, e.g. arranged upstream of the clearing arrangement. The coarse filter may be used to filter larger particles.

According to one variant, the coarse filter is rotatable relative to the device. Such rotatable filter may for example be a cylindrical filter configured to use centripetal forces to force fluid through the filter while keeping substantially dry particles inside the cylinder.

Alternatively, or in addition, the device according to the present disclosure may comprise a labyrinth path fluidly between the suction inlet and the exhaust outlet. The labyrinth path may for example be provided fluidly between the clearing arrangement and the exhaust outlet. The labyrinth path further prevents liquid from reaching the exhaust outlet. In one example, the labyrinth path comprises at least two bends of at least 120 degrees.

According to a further aspect, there is provided a device for an aspirator, the device comprising a suction inlet for suctioned air, liquid and particles; an exhaust outlet for air; a reservoir for collecting liquid and particles separated from the air; and a clearing arrangement fluidly between the suction inlet and the exhaust outlet, the clearing arrangement being configured to provide a path of air, and substantially cleared from liquid, to the exhaust filter. The clearing arrangement may be movable, attachable and/or configured to change in shape as described herein, and/or may be configured to separate liquid from air by means of attraction (e.g. absorption) as described herein.

According to a further aspect, there is provided an aspirator, such as a medical aspirator, comprising a device according to the present disclosure. The device may comprise a vacuum pump, e.g. for suction from the suction inlet and exhaust through the exhaust outlet. In this case, the aspirator may comprise a main part having a motor for driving the vacuum pump. The device may be detachably attached to the main part. Throughout the present disclosure, the aspirator may be handheld. The device according to the present disclosure may be disposable.

According to a further aspect, there is provided a method of modifying a device for an aspirator, such as a medical aspirator, the method comprising providing a device configured to be used in an aspirator, wherein the device comprises a suction inlet for suctioned air, liquid and particles; an exhaust outlet for air; and a reservoir for collecting liquid and particles separated from the air; and adding a clearing arrangement to the device fluidly between the suction inlet and the exhaust outlet, the clearing arrangement being configured to provide a path of air, substantially cleared, or cleared, from liquid sucked through the suction inlet, to the exhaust outlet, in any orientation of the device in space. Thus, an already functionable device can be upgraded with a clearing arrangement according to the present disclosure. The device and the clearing arrangement of the method may be of any type according to the present disclosure. The clearing arrangement may for example be inserted into the reservoir.

### BRIEF DESCRIPTION OF THE DRAWINGS

Further details, advantages and aspects of the present disclosure will become apparent from the following embodiments taken in conjunction with the drawings, wherein:

FIG. 1: schematically represents a perspective view of an aspirator;

FIG. 2: schematically represents a cross-sectional perspective view of the aspirator in FIG. 1;

FIG. 3: schematically represents a cross-sectional side view of the medical aspirator in FIGS. 1 and 2;

FIG. 4a: schematically represents a cross-sectional side view of a medical aspirator;

FIG. 4b: schematically represents a partial view of a clearing arrangement of the aspirator in FIG. 4a;

FIG. 5: schematically represents a cross-sectional side view of a further example of an aspirator;

FIG. 6a: schematically represents a cross-sectional side view of a further example of an aspirator;

FIG. 6b: schematically represents a clearing arrangement of the medical aspirator in FIG. 6a;

FIG. 7: schematically represents a cross-sectional side view of a further example of an aspirator;

FIG. 8: schematically represents a cross-sectional side view of a further example of an aspirator;

FIG. 9: schematically represents a cross-sectional side view of a further example of an aspirator;

FIG. 10: schematically represents a cross-sectional side view of the medical aspirator in FIG. 9;

FIG. 11: schematically represents a cross-sectional side view of a further example of an aspirator;

FIG. 12: schematically represents a cross-sectional side view of a further example of an aspirator;

FIG. 13: schematically represents a cross-sectional side view of a further example of an aspirator;

FIG. 14: schematically represents a cross-sectional side view of the medical aspirator in FIG. 13;

FIG. 15: schematically represents a cross-sectional side view of the medical aspirator in FIGS. 13 and 14;

FIG. 16: schematically represents a cross-sectional side view of a further example of an aspirator;

FIG. 17: schematically represents a cross-sectional side view of a further example of an aspirator; and

FIG. 18: schematically represents a cross-sectional side view of a further example of an aspirator.

### DETAILED DESCRIPTION

In the following, a device for an aspirator, which device comprises a clearing arrangement, an aspirator comprising a device, and a method of modifying a device for an aspirator, will be described. The same reference numerals will be used to denote the same or similar structural features.

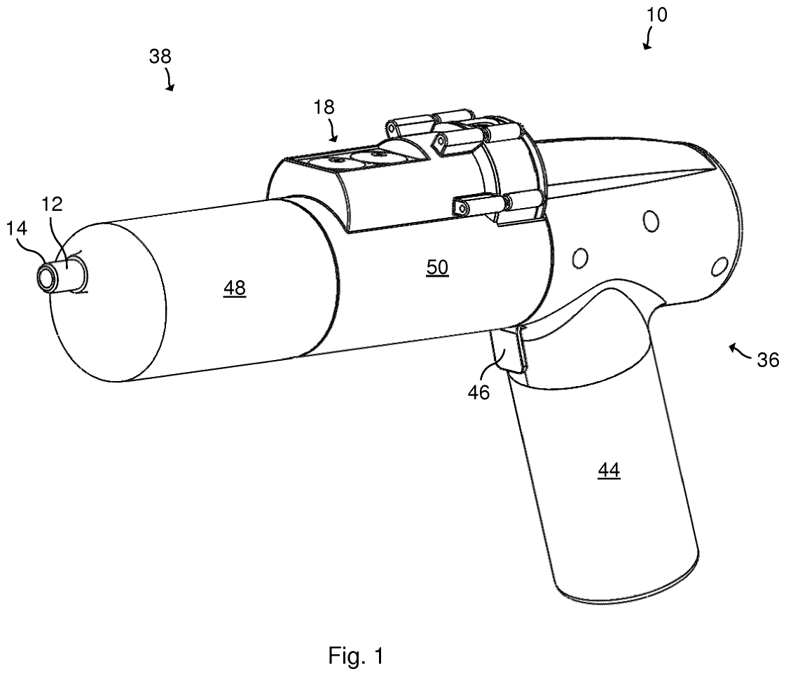

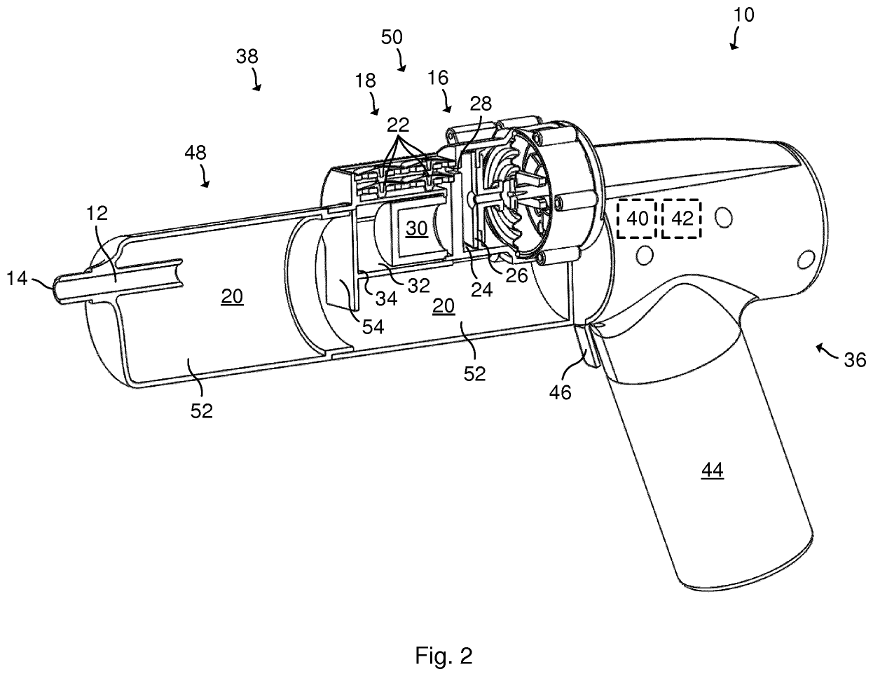

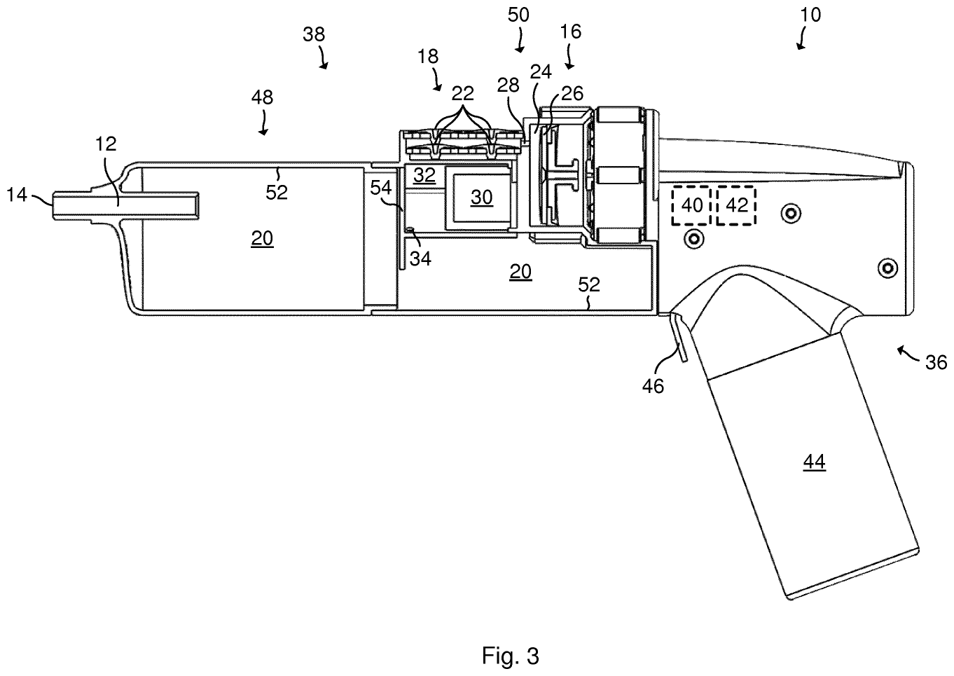

FIG. 1 schematically represents a perspective view of one specific and non-limiting example an aspirator 10, FIG. 2 schematically represents a cross-sectional perspective view of the aspirator 10 in FIG. 2, and FIG. 3 schematically represents a cross-sectional side view of the aspirator 10 in FIGS. 1 and 2. With collective reference to FIGS. 1 to 3, the aspirator 10 comprises a suction inlet 12 having a suction tip 14, a vacuum pump 16 and an exhaust outlet 18. The aspirator 10 further comprises a reservoir 20 fluidly between the suction inlet 12 and the vacuum pump 16. In FIGS. 1 to 3, the aspirator 10 is shown in a horizontal orientation. In the examples in FIGS. 1 to 16, the aspirator 10 is exemplified as a medical aspirator.

The exhaust outlet 18 of the aspirator 10 in FIGS. 1 to 3 comprises a plurality of one-way valves 22. The vacuum pump 16 of this example comprises a piston chamber 24 and a piston 26 reciprocatingly movable within the piston chamber 24. The piston chamber 24 comprises a passage 28 to the valves 22. Alternative types of vacuum pumps may however be used.

The aspirator 10 further comprises a filter 30, such as a hydrophobic HEPA filter. The filter 30 is arranged fluidly between the suction inlet 12 and the exhaust outlet 18, in this example fluidly between the reservoir 20 and the vacuum pump 16. The aspirator 10 comprises a filter chamber 32 housing the filter 30. A plurality of openings 34 is provided in the lower part of the filter chamber 32. The openings 34 establish passages for fluid between the reservoir 20 and the filter chamber 32. A passage (not visible) is also provided between the filter chamber 32 and the piston chamber 24.

In this specific and non-limiting example, the aspirator 10 comprises a main part 36, and a device 38 detachably attached to the main part 36. The device 38 may be disposable and the main part 36 may be reusable. The main part 36 comprises a motor 40 for driving the vacuum pump 16, and a power source 42 for powering the motor 40. The main part 36 further comprises a handgrip 44 and a button 46 for controlling the motor 40 and thereby the vacuum pump 16. The handgrip 44 may be detachably attached to the main part 36.

The device 38 of this specific and non-limiting example comprises a canister part 48 and a pump part 50 connected to each other, e.g. by a snap or screw connection. The canister part 48 and the pump part 50 are here generally cylindrical housings. When connected, interior surfaces 52 of the canister part 48 and the pump part 50 form a continuous volume constituting the reservoir 20 for collecting liquid and particles. The suction inlet 12 is provided in the canister part 48. The filter chamber 32, the vacuum pump 16 and the exhaust outlet 18 are provided in the pump part 50.

The device 38 further comprises a distribution structure 54. The distribution structure 54 is oriented substantially perpendicular to the suction inlet 12. In this example, the distribution structure 54 is constituted by a wall of the filter chamber 32 facing the suction inlet 12. The distribution structure 54 shades the openings 34.

When operating the vacuum pump 16, the underpressure established in the device 38 causes air, liquid and various particles to be sucked through the suction inlet 12, for example with a flow of 30 l/min. When the liquid hits the distribution structure 54, the liquid is crushed into smaller droplets in the reservoir 20. Air and small liquid droplets are sucked through the openings 34, into the filter chamber 32 and to the filter 30. The filter 30 removes the liquid droplets from the air passing therethrough. The air is pumped out through the exhaust outlet 18 by means of the vacuum pump 16.

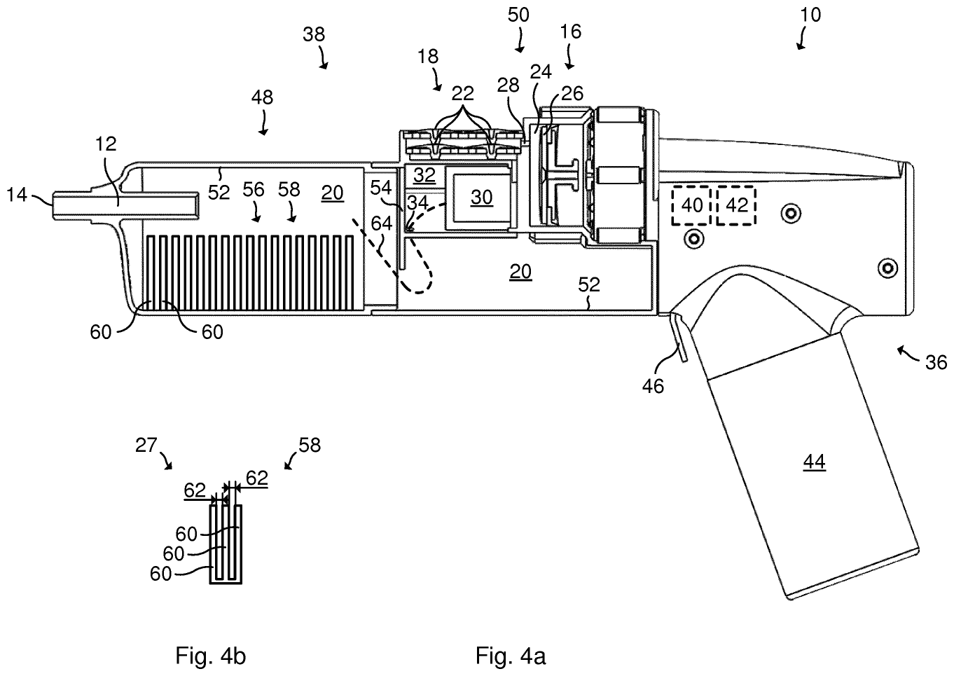

FIG. 4a schematically represents a cross-sectional side view of an aspirator 10 comprising a clearing arrangement 56 and FIG. 4b schematically represents a partial view of the clearing arrangement 56 in FIG. 4a. With collective reference to FIGS. 4a and 4b, the clearing arrangement 56 comprises a capillary structure 58. The capillary structure 58 comprises a plurality of parallel plates 60. A small distance 62 resembling capillaries is provided between each pair of adjacent plates 60. In this example, the plates 6o are attached to the interior surface 52 of the reservoir 20 and protrude radially inwards. As an alternative, each plate 60 may comprise a serrated or wave-formed surface such that elongated capillaries are formed between such adjacent surfaces of the plates 6o when mated. The clearing arrangement 56 in FIG. 4a is thus arranged fluidly between the suction inlet 12 and the exhaust outlet 18, more specifically between the suction inlet 12 and the filter chamber 32.

When liquid has entered the reservoir 20, the liquid, e.g. in the form of liquid droplets and/or larger bodies of liquid, is attracted into the spaces between the plates 60 by means of capillary absorption. Thereby, a path 64 of air, substantially cleared from liquid, is established. The capillary structure 58 can hold liquid in any orientation of the device 38 in space. Thereby, also the path 64 of air can be provided in any orientation of the device 38 in space when the liquid is held by the capillary structure 58. The amount of liquid sucked to the filter 30 is reduced, including when the device 38 is held in a non-horizontal orientation in space. Thereby, clogging of the filter 30 can also be reduced or eliminated, leading to a longer lifetime of the device 38.

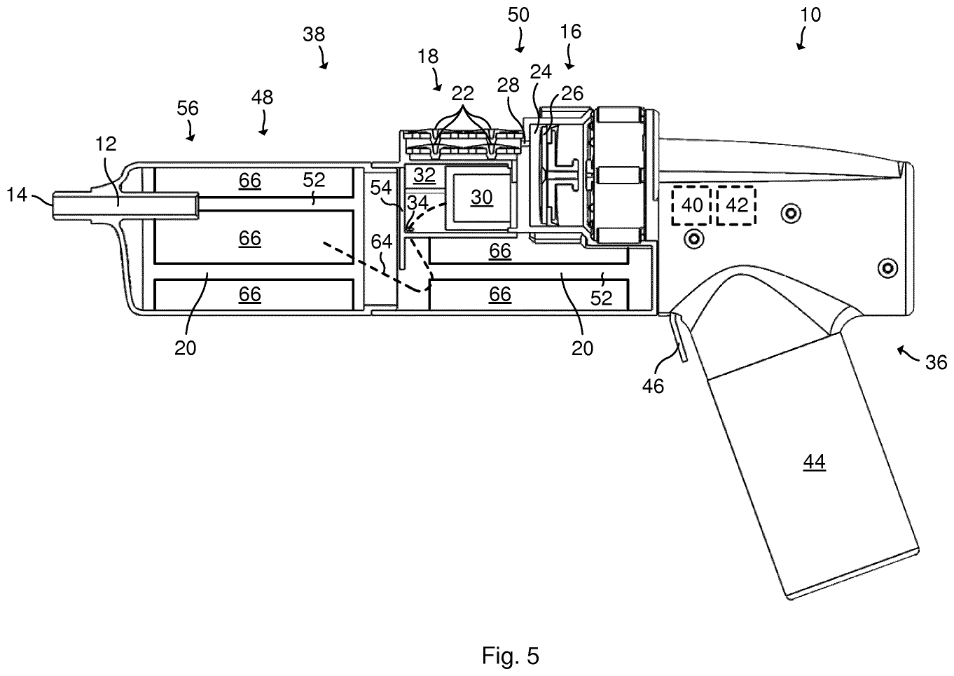

FIG. 5 schematically represents a cross-sectional side view of an aspirator 10 comprising an alternative clearing arrangement 56. Mainly differences with respect to FIGS. 4a and 4b will be described.

The device 38 of the aspirator 10 in FIG. 5 comprises adhesive 66. The adhesive 66 has been provided by attaching strips of double-sided tape to the interior surface 52 of the reservoir 20. In this way, an already functioning aspirator 10 can be modified. The adhesive 66 attracts liquid, either directly or indirectly. The adhesive 66 can for example keep tissue containing liquid to the interior surface 52. Also in this way, a path 64 substantially cleared from liquid can be provided in any orientation of the device 38 in space. The clearing arrangements 56 in FIGS. 4a and 5 may be combined. The adhesive 66 may also be used to hold one or more free bodies according to the present disclosure.

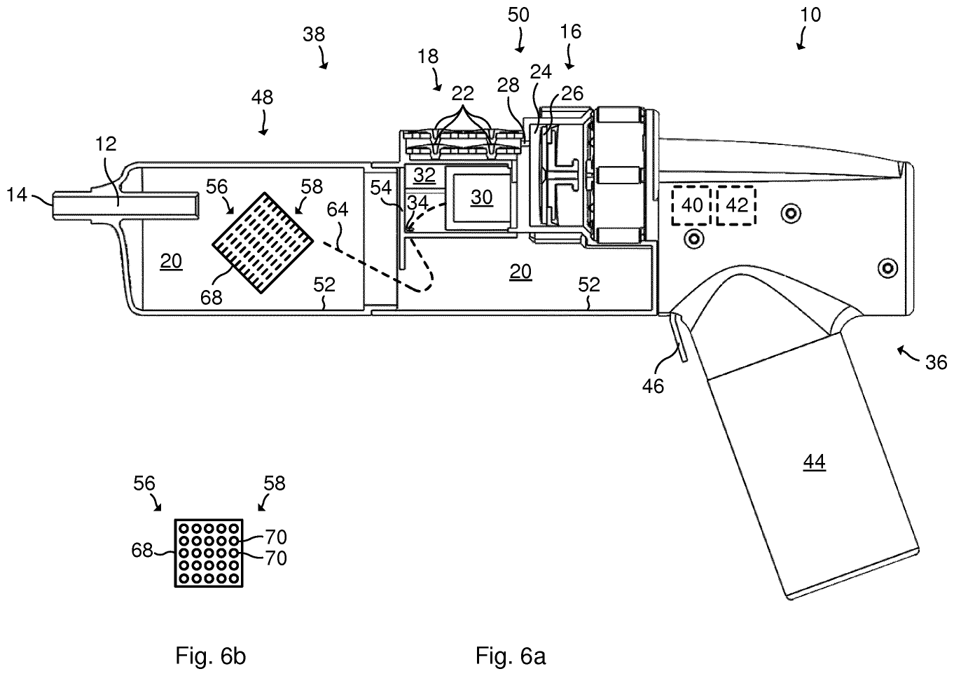

FIG. 6a schematically represents a cross-sectional side view of an aspirator 10 comprising an alternative a clearing arrangement 56 and FIG. 6b schematically represents the clearing arrangement 56 in FIG. 6a. Mainly differences with respect to FIGS. 4a to 5 will be described.

The clearing arrangement 56 in FIG. 6 comprises a free body 68. The free body 68 is movable within the reservoir 20 and constitutes a capillary structure 58. In this example, the free body 68 is constituted by a rigid block comprising a plurality of capillaries 70. Thus, liquid within the reservoir 20 can be attracted by the free body 68 by means of capillary absorption. As can be seen in FIG. 6a, the free body 68 is larger than the openings 34 into the filter chamber 32 and is therefore prevented from entering the filter chamber 32. The free body 68 is also larger than a space between the distribution structure 54 and the interior surface 52. The clearing arrangement 56 in FIGS. 4a, 5 and 6a may be combined, e.g. adhesive 66 may be applied to the free body 68.

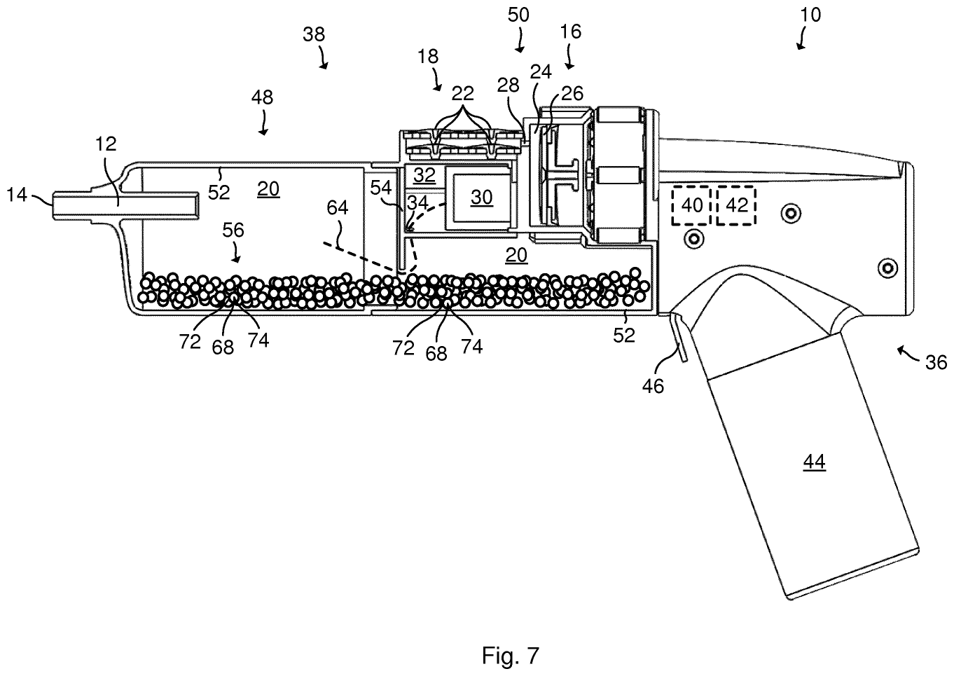

FIG. 7 schematically represents a cross-sectional side view of an aspirator 10 comprising an alternative clearing arrangement 56. Mainly differences with respect to FIGS. 4a to 6b will be described.

The clearing arrangement 56 in FIG. 7 comprises a plurality of free bodies 68, here exemplified as balls. The free bodies 68 are freely movable within the reservoir 20. The clearing arrangement 56 further comprises superabsorbent polymers 72. In this example, the superabsorbent polymers 72 are disposed on the free bodies 68. The free bodies 68 thereby constitute carriers 74 for the superabsorbent polymers 72. The superabsorbent polymers 72 on the free bodies 68 are thereby configured to absorb liquid. Adhesive 66 may also be provided on the free bodies 68. Thereby, the free bodies 68 can adhere to the interior surface 52.

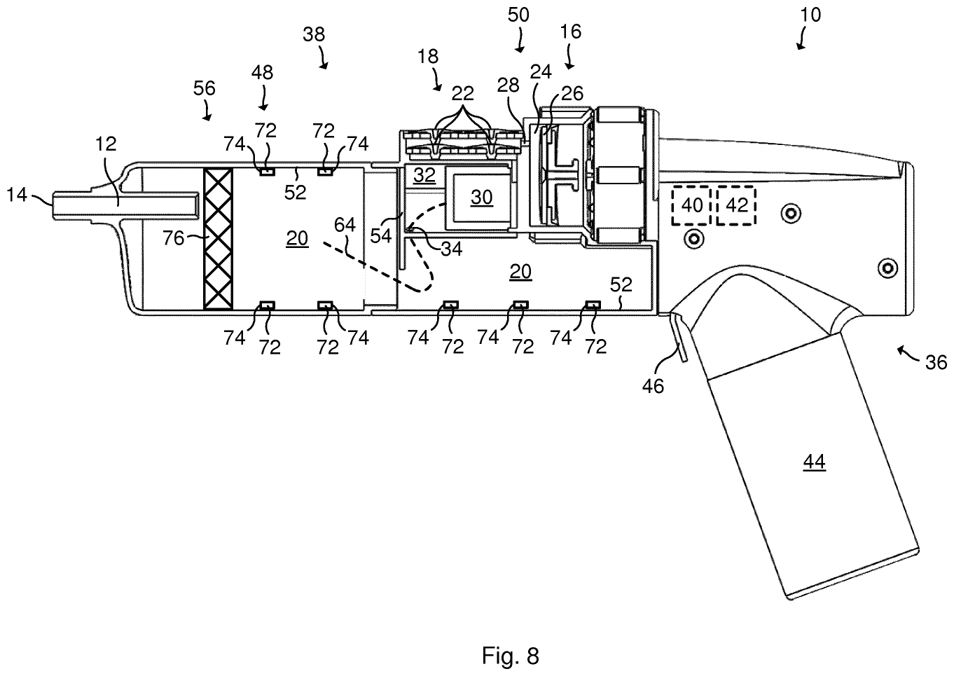

FIG. 8 schematically represents a cross-sectional side view of an aspirator 10 comprising an alternative clearing arrangement 56. Mainly differences with respect to FIGS. 4a to 7 will be described.

The clearing arrangement 56 in FIG. 8 comprises superabsorbent polymers 72. The superabsorbent polymers 72 are disposed in a plurality of carriers 74.

The carriers 74 are liquid permeable and may be constituted by bags or pockets. Moreover, the carriers 74 of this example are attached to the interior surface 52 of the reservoir 20. The carriers 74 may alternatively be freely movable in the device 38, for example as the free bodies 68 in FIG. 7. When liquid contacts the superabsorbent polymers 72 in the carriers 74 in FIG. 8, the superabsorbent polymers 72 absorb the liquid and swell. The liquid can thereby be held to the interior surface 52 of the reservoir 20 (or at any other location of the device 38 where the carriers 74 are provided). As a consequence, the path 64 of air, substantially cleared from liquid, can be provided through the filter 30 and to the exhaust outlet 18. The clearing arrangement 56 comprising superabsorbent polymers 72 according to FIG. 8 may be combined with clearing arrangements 56 based on capillary action and/or adhesive 66. Absorbent materials other than superabsorbent polymers 72, for example silica gel, may be used in the clearing arrangement 56.

The device 38 in FIG. 8 also comprises a coarse filter 76. The coarse filter 76 functions to separate bone fragments, food chunks and similar particles from the suctioned fluid. A mesh size of the coarse filter 76 is preferably dimensioned to be larger than the openings 34. In FIG. 8, the coarse filter 76 is arranged in the reservoir 20 upstream of the clearing arrangement 56.

However, the coarse filter 76 may alternatively be arranged downstream of the clearing arrangement 56 or in parallel with the clearing arrangement 56.

A coarse filter 76 of the type in FIG. 8 may be provided to the device 38 in any of the remaining figures.

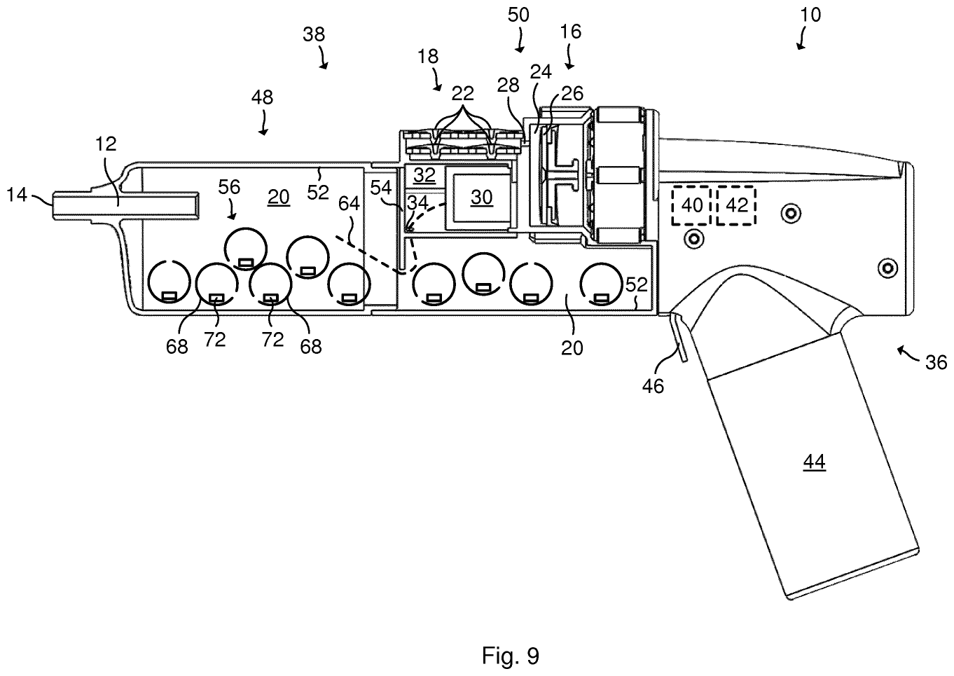

FIG. 9 schematically represents a cross-sectional side view of an aspirator 10 comprising an alternative clearing arrangement 56. Mainly differences with respect to FIGS. 4a to 8 will be described.

The clearing arrangement 56 in FIG. 9 comprises a plurality of free bodies 68 within the reservoir 20. Each free body 68 contains superabsorbent polymers 72, for example provided in the form of a carrier 74 according to FIG. 8. The free bodies 68 in FIG. 9 may be rigid. Each free body 68 is permeable by comprising at least one opening. Thereby, liquid can enter the free bodies 68 and come in contact with the superabsorbent polymers 72 at the same time as larger particles are prevented from entering the free bodies 68. The free bodies 68 move together with any liquid in the reservoir 20 due to gravity. This improves liquid absorption by means of the superabsorbent polymers 72. According to one example, a coarse filter 76 (see FIG. 9) is fluidly between the suction inlet 12 and the clearing arrangement 56. Bone fragments and food chunks may thereby be collected in the coarse filter 76, while fluid is allowed to pass through the coarse filter 76 and to the free bodies 68 provided either in the reservoir 20 or in another compartment.

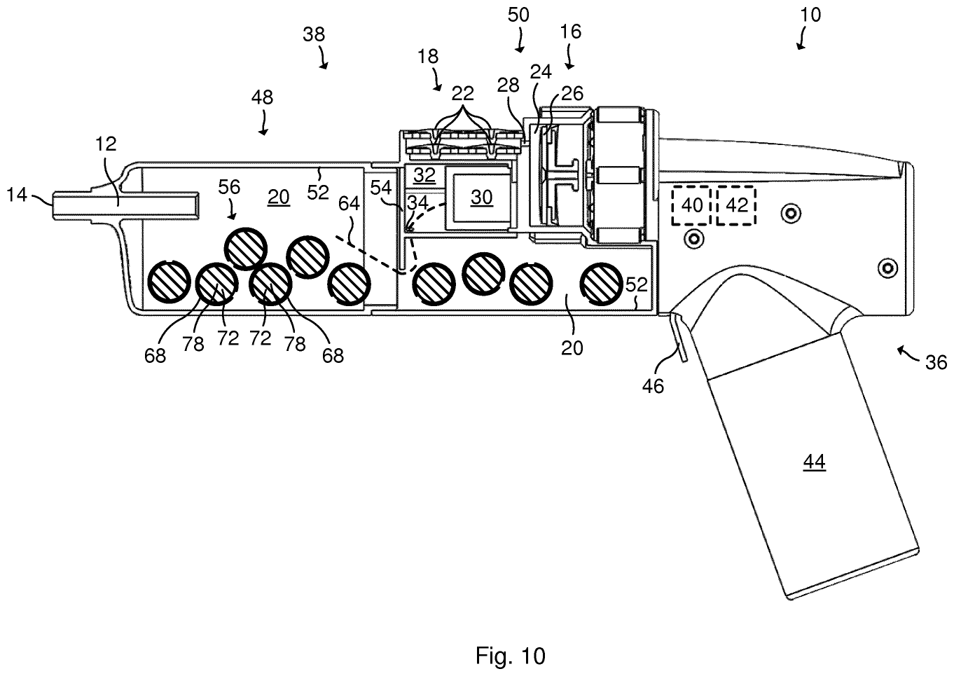

FIG. 10 schematically represents a cross-sectional side view of the aspirator 10 in FIG. 9. As shown in FIG. 10, the superabsorbent polymers 72 within the free bodies 68 have absorbed liquid 78. The free bodies 68 are larger than the openings 34 into the filter chamber 32 and are therefore prevented from entering the filter chamber 32. Moreover, due to the relatively large size of the free bodies 68, the superabsorbent polymers 72 can attract a relatively large amount of liquid 78 while providing the path 64 of air in interstices between free bodies 68 and/or between free bodies 68 and the interior surface 52. The clearing arrangement 56 in FIGS. 9 and 10 may additionally comprise adhesive 66 and/or a capillary structure 58.

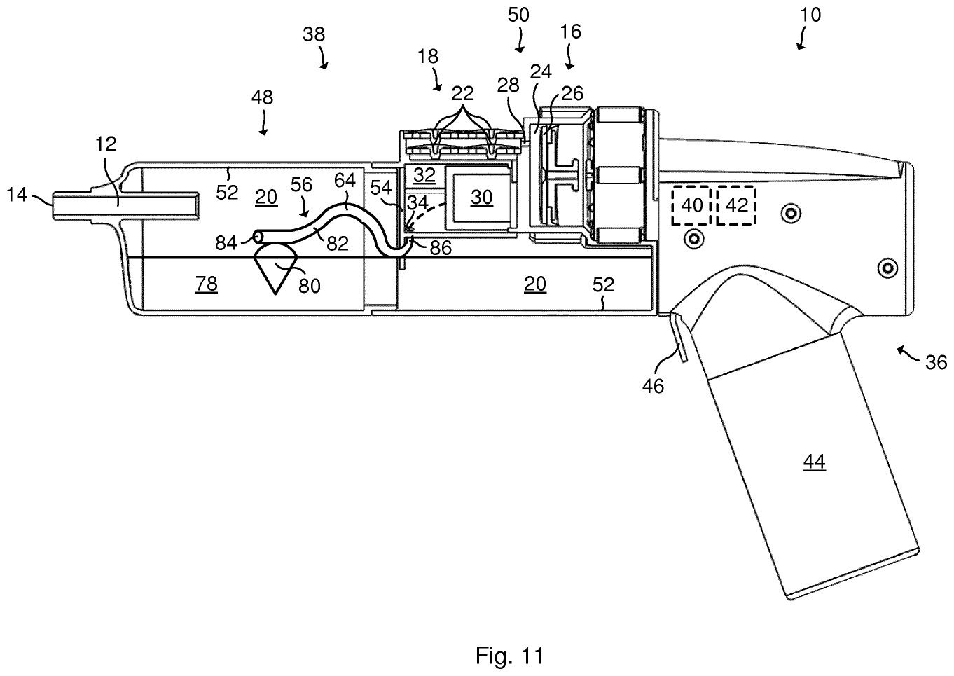

FIG. 11 schematically represents a cross-sectional side view of an aspirator 10 comprising an alternative clearing arrangement 56. Mainly differences with respect to FIGS. 4a and 10 will be described.

The clearing arrangement 56 in FIG. 11 comprises a floating element 80 and a tube 82. The tube 82 comprises a tube inlet 84 and a tube outlet 86. The tube 82 is connected to the floating element 80 such that the tube inlet 84 is above (i.e. geodetically above) the floating element 80. The floating element 8o is configured to substantially maintain the vertical orientation in FIG. 11 when floating on the surface of the liquid 78. In FIG. 11, only one opening 34 is provided into the filter chamber 32. The tube outlet 86 is tightly connected to this opening 34. Thus, the only way for air to enter the filter chamber 32 is through the path 64 provided by the tube 82. Since the floating element 80 ensures that the tube inlet 84 is always above the surface of the liquid 78, the clearing arrangement 56 provides the path 64 of air, substantially free of liquid 78 to the exhaust outlet 18. The clearing arrangement 56 of the type in FIG. 11 may comprise a plurality of floating elements 80 and tubes 82, for example one tube 82 for each of a plurality of openings 34. The clearing arrangement 56 in FIG. 11 may be combined with clearing arrangements 56 comprising an adhesive 66, capillary structures 58 and/or absorbing materials.

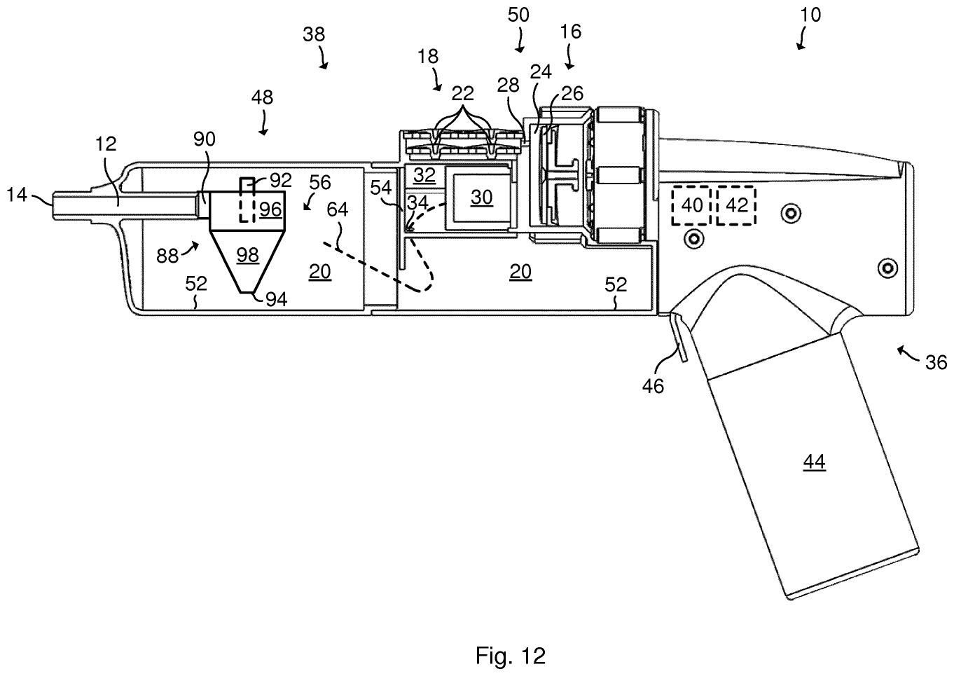

FIG. 12 schematically represents a cross-sectional side view of an aspirator 10 comprising an alternative clearing arrangement 56. Mainly differences with respect to FIGS. 4a to 11 will be described.

The clearing arrangement 56 in FIG. 12 comprises a cyclone separator 88 arranged in the reservoir 20. The cyclone separator 88 of the example in FIG. 12 comprises an inlet 90, an upper (in FIG. 12) air outlet 92, a lower liquid outlet 94, an upper cylindrical portion 96 and a lower frustoconical portion 98. The inlet 90 of the cyclone separator 88 is tightly connected to the suction inlet 12. During operation of the aspirator 10, suctioned air, liquid and particles enter through the inlet 90, which is positioned substantially tangential to an inner surface of the upper cylindrical portion 96. The shapes of the upper cylindrical portion 96 and the lower frustoconical portion 98 induce a vortex by means of which liquid and particles are forced out through the lower liquid outlet 94 and air is forced out through the upper air outlet 92. Thereby, also the clearing arrangement 56 in FIG. 12 is configured to provide the path 64 substantially cleared from liquid.

According to one modification, the cyclone separator 88 arranged to move in response to the gravity force, e.g. to maintain a vertical orientation. One way to accomplish this is to connect the inlet 90 of the cyclone separator 88 to the suction inlet 12 by means of a swivel coupling. In this manner, it can be ensured that the air outlet 92 is always geodetically above the liquid outlet 94.

The cyclone separator 88 may alternatively be arranged upstream of the reservoir 20. The clearing arrangement 56 comprising a cyclone separator 88 may be combined with any other clearing arrangement 56 as described herein.

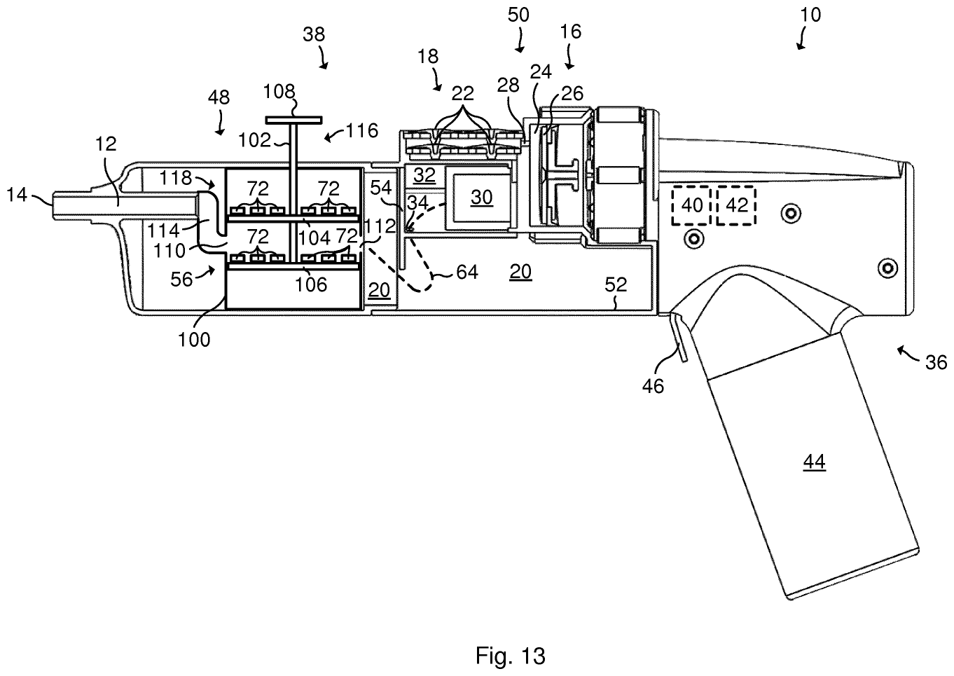

FIG. 13 schematically represents a cross-sectional side view of an aspirator 10 comprising an alternative device 38. Mainly differences with respect to FIGS. 4a to 12 will be described.

The clearing arrangement 56 in FIG. 13 comprises superabsorbent polymers 72. The device 38 in FIG. 13 comprises a piston chamber 100 within the reservoir 20. The device 38 further comprises a piston rod 102, and a first piston 104 and a second piston 106 fixed to the piston rod 102. The first piston 104 and the second piston 106 are movable within the piston chamber 100. The piston rod 102 protrudes through the reservoir 20 and to the exterior of the device 38 where a button 108 is attached to the piston rod 102.

The piston chamber 100 comprises an inlet no and an outlet 112. A connection tube 114 connects the suction inlet 12 to the inlet no. The outlet 112 is open into the reservoir 20. The piston chamber 100, the piston rod 102, the first piston 104, the second piston 106 and the button 108 merely constitute one of many examples of a switching mechanism 116 according to the present disclosure.

As shown in FIG. 13, the clearing arrangement 56 comprising superabsorbent polymers 72 is provided between the first piston 104 and the second piston 106. Furthermore, the inlet no and the outlet 112 are positioned between the first piston 104 and the second piston 106. The device 38 further comprises a secondary clearing arrangement 118, here exemplified as comprising superabsorbent polymers 72. In FIG. 13, the secondary clearing arrangement 118 is provided within the piston chamber 100 above the first piston 104 and is thereby in an inactive state, where the superabsorbent polymers 72 are fluidly disconnected from the suction inlet 12. In operation, air, liquid and any particles can be sucked past the clearing arrangement 56 causing the superabsorbent polymers 72 to absorb any liquid passing by. However, the superabsorbent polymers 72 of the secondary clearing arrangement 118 will remain isolated.

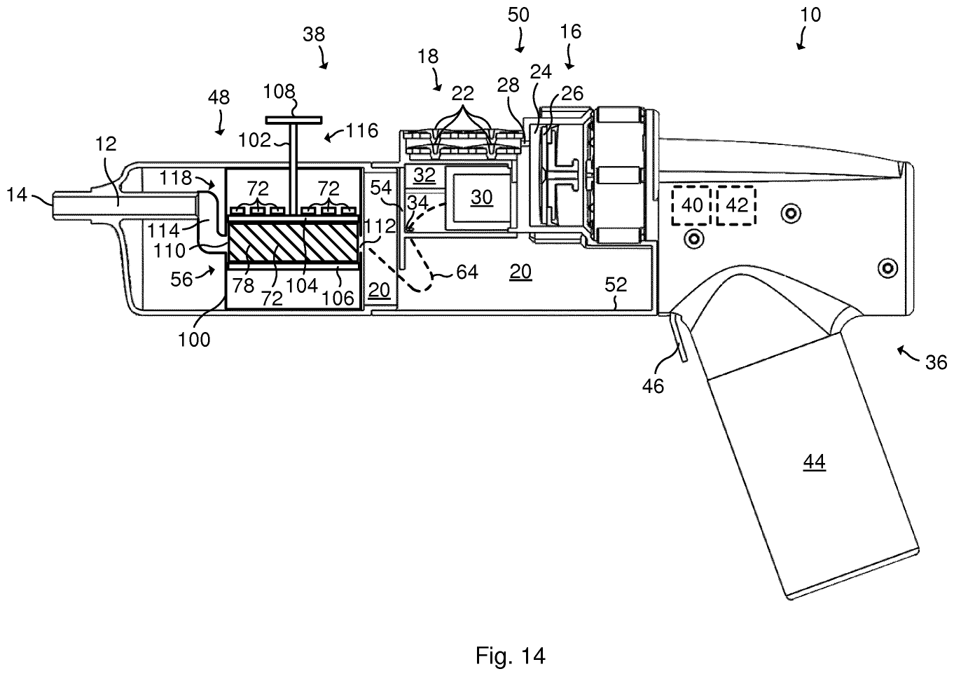

After some time of operation of the aspirator 10, the superabsorbent polymers 72 of the clearing arrangement 56 have swelled, as shown in FIG. 14.

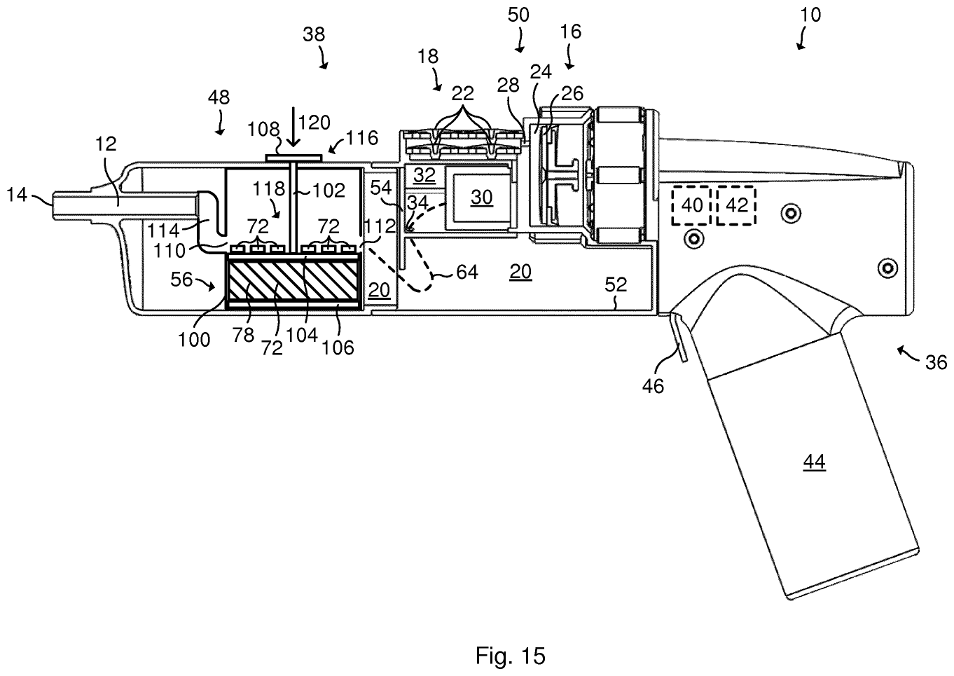

Instead of interrupting a suction operation or replacing the device 38 or the entire aspirator 10, a user may simply push down the button 108 as indicated by arrow 120 in FIG. 15. Thereby, the first piston 104 will move below the inlet 110 and the outlet 112 and expose the unused superabsorbent polymers 72 of the secondary clearing arrangement 118 to incoming liquid (and air and any particles). By means of the switching mechanism 116, the secondary clearing arrangement 118 has thereby adopted an active state in which the secondary clearing arrangement 118 is configured to provide the path 64 of air substantially cleared from liquid. In this way, the lifetime of the device 38 is substantially extended in a simple, reliable and fast way.

Although the secondary clearing arrangement 118 has been described as comprising superabsorbent polymers 72, the secondary clearing arrangement 118 may be of any type as the clearing arrangement 56 according to the present disclosure. In particular, the secondary clearing arrangement 118 may alternatively, or additionally, comprise a capillary structure 58, adhesive 66 and/or absorbing materials other than superabsorbent polymers 72.

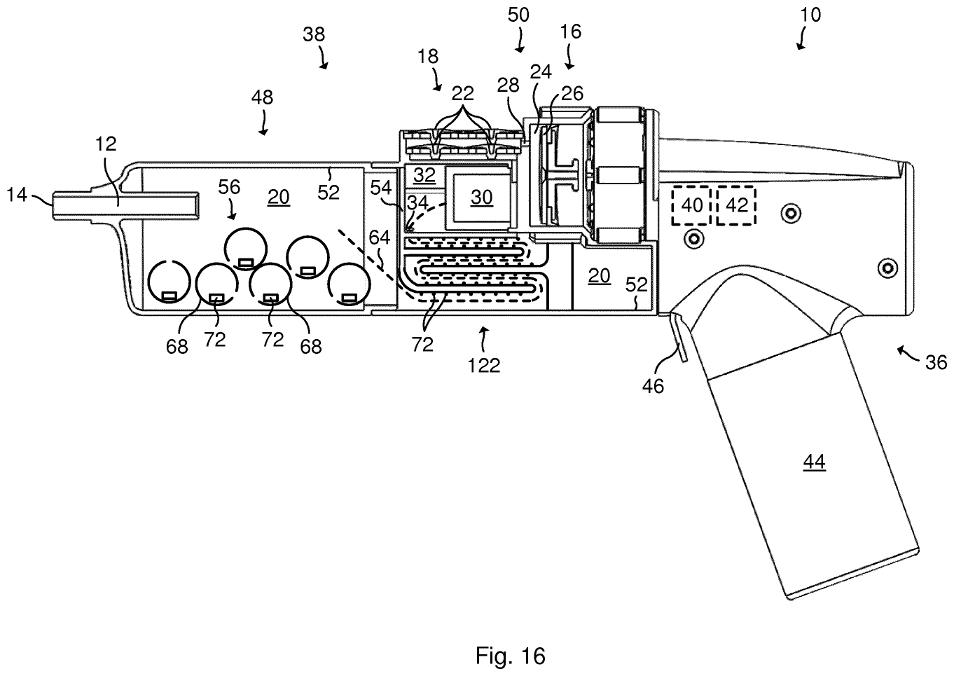

FIG. 16 schematically represents a cross-sectional side view of a further example of an aspirator 10. The device 38 in FIG. 16 comprises the same type of clearing arrangement 56 as in FIG. 9. The device 38 further comprises a labyrinth path 122. The labyrinth path 122 is arranged fluidly between the suction inlet 12 and the exhaust outlet 18. More specifically, the labyrinth path 122 is arranged in the reservoir 20 immediately upstream of the filter chamber 32. The labyrinth path 122 may however alternatively be provided outside the reservoir 20, such as upstream from, downstream from, or parallel with, the reservoir 20. The labyrinth path 122 in of the specific example in FIG. 16 comprises three bends of approximately 180 degrees and one bend into the opening 34 of the filter chamber 32 of approximately 90 degrees. In FIG. 16, superabsorbent polymers 72 are provided on the walls of the labyrinth path 122, e.g. in small amounts such that the labyrinth path 122 cannot close due to the swelling of the superabsorbent polymers 72. The labyrinth path 122 thereby further prevents potential remaining liquid from entering the filter 30.

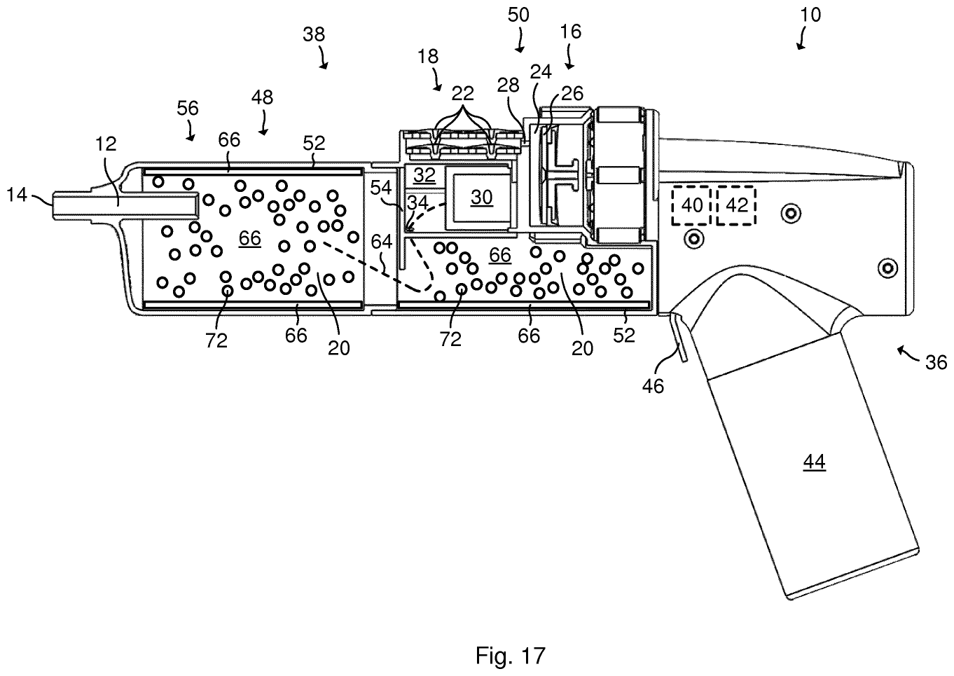

FIG. 17 schematically represents a cross-sectional side view of a further example of an aspirator 10. Mainly differences with respect to FIGS. 4a to 16 will be described. The clearing arrangement 56 of the aspirator 10 in FIG. 17 comprises adhesive 66. Furthermore, the clearing arrangement 56 comprises a plurality of free bodies 68, such as balls.

The device 38 in FIG. 17 has been modified by first providing the adhesive 66, e.g. by spraying, onto the inner surface 52, and then blowing absorbent material, such as superabsorbent polymers 72, into the reservoir 20. The superabsorbent polymers 72 may for example be delivered into the reservoir 20 in powder form and/or in carriers 74. Alternatively, or in addition, the superabsorbent polymers 72 may be delivered into the reservoir 20 together with the adhesive 66. The superabsorbent polymers 72 thereby stick to the inner surface 52. Any liquid absorbed by the superabsorbent polymers 72 will thereby cause swelling of the superabsorbent polymers 72 in a radially outer region of the reservoir 20, i.e. at the interior surface 52. Also in this way, a path 64 substantially cleared from liquid can be provided in any orientation of the device 38 in space.

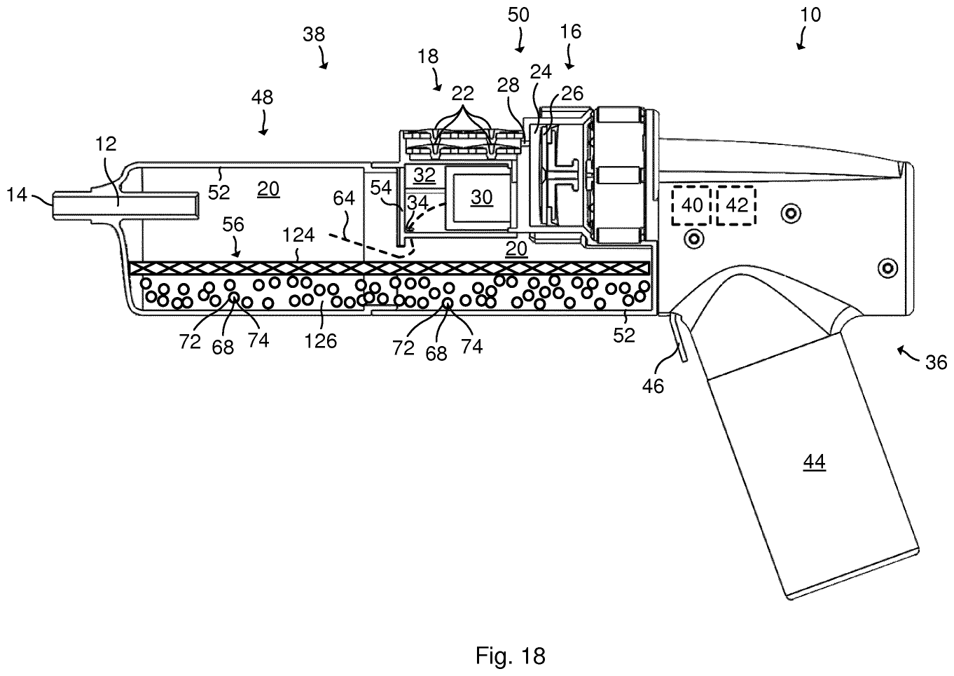

FIG. 18 schematically represents a cross-sectional side view of a further example of an aspirator 10. Mainly differences with respect to FIGS. 4a to 17 will be described. The device 38 in FIG. 18 comprises a partition wall 124. The partition wall 124 defines a partition volume 126. In the example of FIG. 18, the partition wall 124 and the partition volume 126 are provided within the reservoir 20. Furthermore, the partition wall 124 of this example comprises a coarse filter, e.g. of the same type as in FIG. 8.

The clearing arrangement 56 in FIG. 18 comprises a plurality of free bodies 68 of the same type as in FIG. 7. The free bodies 68 are provided within the partition volume 126. The partition wall 124 maintains the free bodies 68 within the partition volume 126. Any liquid within the reservoir 20 will permeate the partition wall 124 and enter the partition volume 126. Coarse particles are prevented from entering the partition volume 126. Thereby, the attraction of liquid by the clearing arrangement 56, e.g. by means of absorption by the superabsorbent polymers 72 or by means of capillary action, is improved. Furthermore, the swelling of the superabsorbent polymers 72 can be substantially limited within the partition volume 126.

Larger particles that cannot enter the partition volume 126 also cannot enter the filter chamber 32 due to the size of the openings 34.

While the present disclosure has been described with reference to exemplary embodiments, it will be appreciated that the present invention is not limited to what has been described above. For example, it will be appreciated that the dimensions of the parts may be varied as needed.

1. A device for an aspirator, the device comprising:
a suction inlet for suctioned air, liquid and particles;
an exhaust outlet for air;
a reservoir for collecting liquid and particles separated from the air; and
a clearing arrangement fluidly between the suction inlet and the exhaust outlet, the clearing arrangement being configured to provide a path of air, substantially cleared from liquid sucked through the suction inlet, to the exhaust outlet, in any orientation of the device in space.

2. The device according to claim 1, further comprising a filter fluidly between the suction inlet and the exhaust outlet; wherein the clearing arrangement is fluidly between the suction inlet and the filter, and wherein the clearing arrangement is configured to provide a path of air, substantially cleared from liquid sucked through the suction inlet, to the filter, in any orientation of the device in space.

3-4. (canceled)

5. The device according to claim 1, wherein the clearing arrangement is configured to separate liquid from air by means of absorption, and wherein the clearinq arrangement comprises an absorbent material.

6-9. (canceled)

10. The device according to claim 1, wherein the clearing arrangement comprises a plurality of free bodies.

11. The device according to claim 1, wherein the clearing arrangement is positioned within the reservoir.

12. The device according to claim 11, wherein the clearing arrangement comprises a floating element configured to float on collected liquid within the reservoir, and a tube having a tube inlet and a tube outlet, and wherein the tube inlet is connected to the floating element and the tube outlet is arranged downstream along the path.

13. The device according to claim 1, further comprising a secondary clearing arrangement and a switching mechanism; wherein the switching mechanism is configured to switch the secondary clearing arrangement from an inactive state, in which the secondary clearing arrangement is fluidly disconnected from the suction inlet, to an active state, in which the secondary clearing arrangement is arranged fluidly between the suction inlet and the exhaust outlet, and wherein the secondary clearing arrangement is configured to provide a path of air, substantially cleared from liquid sucked through the suction inlet, to the exhaust outlet, in any orientation of the device in space.

14. An aspirator comprising a device according to claim 1.

15. The aspirator according to claim 14, wherein the device comprises a vacuum pump, and wherein the aspirator comprises a main part having a motor for driving the vacuum pump.

16. The aspirator according to claim 14, wherein the aspirator is handheld.

17. A method of modifying a device for an aspirator, the method comprising:
providing a device configured to be used in an aspirator, wherein the device comprises a suction inlet for suctioned air, liquid and particles; an exhaust outlet for air; and a reservoir for collecting liquid and particles separated from the air; and
adding a clearing arrangement to the device fluidly between the suction inlet and the exhaust outlet, the clearing arrangement being configured to provide a path of air, substantially cleared from liquid sucked through the suction inlet, to the exhaust outlet, in any orientation of the device in space.

18. The device according to claim 1, further comprising a distribution structure configured to distribute suctioned liquid into smaller droplets, wherein the distribution structure is constituted by a wall.

19. The device according to claim 2, further comprising a filter chamber, wherein the filter is housed within the filter chamber and comprises one or more passages for providing air communication between the reservoir and the filter chamber, and wherein the distribution structure shades the one or more passages.

20. The device according to claim 19, wherein the distribution structure is constituted by a wall of the filter chamber.

21. The device according to claim 18, wherein the distribution structure is constituted by a wall substantially perpendicular to a center axis of a discharge opening of the suction inlet.

22. The device according to claim 5, further comprising a partition wall defining a partition volume, wherein the absorbent material is provided within the partition volume.

23. The device according to claim 10, wherein the free bodies are movable within the reservoir.

24. The device according to claim 5, wherein the clearing arrangement comprises a plurality of free bodies, and wherein the absorbent material is provided in or on the free bodies.

25. The device according to claim 1, further comprising a labyrinth path fluidly between the suction inlet and the exhaust outlet.

26. The device according to claim 2, further comprising a labyrinth path fluidly between the suction inlet and the exhaust outlet, wherein the labyrinth path is provided fluidly between the clearing arrangement and a filter chamber, wherein the filter is housed within the filter chamber and comprises one or more passages for providing air communication between the reservoir and the filter chamber, and wherein the distribution structure shades the one or more passages.
